(19) United States
(12) Patent Application Publication
(10) Pub. No.: US 2010/0261795 A1
(43) Pub. Date: Oct. 14, 2010

(54) Antiacneic compositions

(75) Inventor: Herve Buzot, Chatham, NJ (US)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE DERMOCOSMETIQUE USA, Parsippany, NJ (US)

(21) Appl. No.: 12/384,710

(22) Filed: Apr. 8, 2009

(51) Int. Cl. A61K 31/192; A61P 17/10

(52) U.S. Cl. 514/568

(57) ABSTRACT

The invention relates to novel powerful and non-irritating formulations of antiacneic substances and polyolprepolymers. The invention further relates to novel formulations of polyolprepolymers in combination with, for example, benzoyl peroxide exhibit progressive release of the active with a decrease in irritating effects. These formulations may be therapeutically beneficial over existing acne therapies.

### FIELD OF THE INVENTION

The present invention relates to a novel powerful and non-irritating formulations of antiacneic substances and polyolprepolymers and methods of using the same in the treatment of acne.

### BACKGROUND OF THE INVENTION

Acne is a major health and psychological concern, particularly among the adolescent population. Arising primarily out of blockages in hair follicles and their associated sebaceous gland, the pilosebaceous unit, acne manifests bacterial infections in an accumulation of sebum and dead cells. The resulting irritation and inflammation results in what are commonly referred to as pimples, blemishes or more generically, acne.

Numerous causes have been associated with acne development, including hygiene, diet, heredity, and hormonal changes. Acne therapy focuses on the associated bacterial infection and inflammation generated by this accumulation of sebum and dead cells in the effected follicles. Typically therapy occurs through topical administration of actives which prevent or treat the cell proliferation, bacterial infection and resulting inflammation. These may be retinoids which are administered to treat hyperproliferation, bactericidal compositions, and antibiotics. Actives may be administerd by topical and oral routes.

Retinoid substances are typically derivatives of Vitamin A. Derivatives include retinol, isoretretinoin, tretinoin, adapalene, and tazarotene. Bactericidal substances include benzoyl peroxide, triclosan, chlorhexidine gluconate, as well as salicylic acid. Antibiotics include erythromycin, clindamycin, stievamycin, and tetracycline.

Both topical and oral administration of the various actives have issues regarding delivery and side effects. With respect to topical administration, the active may be formulated in everything from soaps and washes to gels and creams. Anti-acne treatment formulation difficulties include side effects such as potential irritation, as well as problems providing the active in a form in which it may effectively be administered to the effected area, providing the active in an effective concentration, and formulating the active such that it is resident on the effected area for a sufficient period of time to affect treatment.

Excipients which are gaining acceptance in formulating actives for topical administration are polyolprepolymers. Polyolprepolymers are a mixture of oligomers ranging from 725 to 5,000 daltons. When applied to the skin, the higher molecular weight polyolprepolymer materials stay on the surface of the skin, while lower weight materials such as cosmetic agents and actives, slowed by the formation of the polyolprepolymer film deposition, differ in their penetration into the stratum corneum. This long lasting cosmetic agent and active release effect is known to work with cosmetic agents or actives that are in soluble form (i.e. salicylic acid) where the stratum corneum penetration and molecule gradient release can effectively take place. This association, however, is typically not known to work with actives or cosmetic agents that are non-soluble, i.e., found in powder or crystal form. Polyolprepolymers form a liquid matrix of polymers in association with the stratum corneum and form long-lasting liquid reservoirs, depositing and holding cosmetic agents on and in the stratum corneum and epidermis. Advantageous properties of polyolprepolymers include their cosmetic elegance, their ability to incorporate into the skin, their resistance to wash-off, the fact that they are non-comedogenic, non-sensitizing and non-irritating, and the fact that they don't block transepidermal water loss, allowing the skin to function normally. Polyolprepolymers were first described in U.S. Pat. No. 4,971,800, filed 18 Sep. 1989 in the name of the Regents of the University of California, claiming the benefit of a number of priority applications dating from as early as 16 Jul. 1987. Polyolprepolymers are commercially available from Barnet Products Corporation, 140 Sylvan Avenue, Englewood Cliffs N.J. 07632.

As representative of less common compositions comprising polyolprepolymers associated with crystal powder non-soluble anti-acne actives, we note those compositions described in US Published Application US 2008/0253986, which application describes a topically applicable, stable cosmetic/dermatological composition which comprises at least one naphthoic acid compound useful for the treatment of keratinization disorders and an anti-irritant amount of at least one polyolprepolymer. The utility of the polyolprepolymer is described as being to increase topical penetration into the epidermis and stratum corneum, to limit desquamation, irritation and dryness of the skin. (See paragraph 0018.) The specification also explains that the polyolprepolymers are demonstrated to both promote the penetration of insoluble and/or dispersed crystalline compounds, but also maintain the anti-irritant effect even when formulating undissolved dispersed crystals.

Clearly, the function of polyolprepolymers in increasing the penetration of actives is dependent on the actives. That composition described in US Published Application US 2008/0253986 comprises naphthoic acid derivatives, such as adapalene, a retinoate, and are described as being present in an insoluble form, dispersed in the composition. Retinoates, such as adapalene, may be be distinguished from other non-soluble antiacneics for their keratolytic activity, which activity is exhibited, for example, by benzoyl peroxide, but the retinoates don't possess the antibacterial activity of, for example, benzoyl peroxide. See ‘Keratolytic’ Properties of Benzoyl Peroxide and Retinoic Acid Resemble Salicylic in Man, J. M. Waller, F. Dreher, S. Behnam, C. Ford, C. Lee, G. D. Weinstein, H. I. Maibach, Skin Pharmacol Phusiol, 19:283-289 (2006).

Another technique which may be employed to enhance penetration is that of solubilizing the benzoyl peroxide. In theory, solubilization facilitates both dispersion as well as penetration into the effected pores. A representative example of such technique is the product OBAGI CLENZIderm M.D.™, which is a commercial 5% liquefied benzoyl peroxide composition wherein the benzoyl peroxide is solubilized down to 10⁻⁷ mm, which homogeneous solution is alleged to provide for better follicular penetration of the active.

### THE PRESENT INVENTION

We have discovered novel powerful and non-irritating formulations of antiacneic substances and polyolprepolymers. The instant novel formulations of polyolprepolymers in combination with, for example, benzoyl peroxide exhibit progressive release of the active with a decrease in irritating effects. Therefore, these formulations may be therapeutically beneficial over existing acne therapies.

### OBJECTS OF THE INVENTION

It is an object of the present invention to provide novel powerful and non-irritating formulations of antiacneic substances and polyolprepolymers. It is a further object to provide a novel method of treating, eliminating, alleviating, palliating or ameliorating undesirable skin disorders, including acne, by administering such formulations.

Yet additional objects will become apparent hereinafter, and still further objects will be apparent to one skilled in the art.

### SUMMARY OF THE INVENTION

What we therefore believe to be comprised by our invention may be summarized inter alia in the following words:

A topical biologically active formulation, comprising a biologically active agent in combination with a penetration enhancer; wherein the biologically active agent is an antiacneic substance; and wherein the penetration enhancer is a polyolprepolymer.

Such a topical biologically active formulation, wherein the antiacneic substance is benzoyl peroxide.

Such a topical biologically active formulation, wherein the antiacneic substance is benzoyl peroxide which has been micronized to 5 μm.

Such a topical biologically active formulation, wherein the polyolprepolymer is polyolpreplymer-2.

Such a topical biologically active formulation, further comprising another substance effective in preventing and treating acne.

A pharmaceutical composition, comprising the topical biologically active formulation, alone or in combination with one or more pharmaceutically acceptable additives and/or excipients.

Such a pharmaceutical composition, wherein the antiacneic substance is benzoyl peroxide.

Such a pharmaceutical composition, wherein the benzoyl peroxide is present in an amount between 0.1 and 20 weight percent.

Such a pharmaceutical composition, wherein the benzoyl peroxide is present in an amount between 3 and 10 weight percent.

Such a pharmaceutical composition, wherein the penetration enhancing agent is a polyolprepolymer.

Such a pharmaceutical composition, wherein the polyolprepolymer is polyolprepolymer-2.

Such a pharmaceutical composition, wherein the polyolprepolymer-2 is present in an amount between 0.1 and 20 weight percent.

Such a pharmaceutical composition, wherein the polyolprepolymer-2 is present in an amount between 1 and 10 weight percent.

Such a pharmaceutical composition, wherein the antiacneic substance is benzoyl peroxide which is present at about 5 weight percent and the polyolprepolymer is polyolprepolymer-2 which is present at about 1 weight percent.

Such a pharmaceutical composition, wherein the composition is in the form of a liquid, gel, lotion, cream or ointment.

The use of the topical biologically active formulation, for the manufacture of a medicament for treatment or prevention of acne.

Such a use of the topical biologically active formulation, wherein the biologically active agent is benzoyl peroxide, for the manufacture of a medicament for treatment or prevention of acne.

Such a use of the topical biologically active formulation, wherein the penetration enhancer is polyolprepolymer-2, for the manufacture of a medicament for treatment or prevention of acne.

A method of treating or preventing acne, comprising administering to a patient in need of such treatment the topical biologically active formulation, which provides the biologically active agent in a therapeutically effective amount.

Such a method, wherein the biologically active formulation comprises a therapeutically active amount of benzoyl peroxide.

Such a method, wherein the biologically active formulation comprises a therapeutically active amount of benzoyl peroxide in combination with polyolprepolymer-2 as penetration enhancer.

### DETAILED DESCRIPTION OF THE INVENTION

### Pharmaceutical Compositions

The term “excipient” applied to pharmaceutical compositions of the invention refers to an adjuvant, carrier, diluent, or vehicle with which the biologically active formulation of the present invention is administered. Such pharmaceutical excipients may be sterile or non-sterile excipients commonly used for the formulation and production of semi solid, liquid and sterile pharmaceutical compositions. These excipients may also be liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. A. R. Gennaro, 20^{th} Edition, describes suitable pharmaceutical carriers in “Remington: The Science and Practice of Pharmacy”. The excipients may also be combinations of solids and liquids.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

### Method of Treating

Due to their high degree of activity and their low toxicity, together presenting a most favorable therapeutic index, the biologically active formulation of the invention may be administered to a subject, e.g., a living animal (including a human) body, in need thereof, for the treatment, alleviation, or amelioration, palliation, or elimination of an indication or condition which is susceptible thereto, or representatively of an indication or condition set forth elsewhere in this application, including concurrently, simultaneously, or together with one or more pharmaceutically-acceptable excipients, carriers, or diluents, including in the form of a pharmaceutical composition thereof, in an effective amount. Suitable dosage ranges are 1-1000 milligrams daily, optionally 10-500 milligrams daily, and optionally 50-500 milligrams daily, depending as usual upon the exact mode of administration, form in which administered, the indication toward which the administration is directed, the subject involved and the body weight of the subject involved, and the preference and experience of the physician or veterinarian in charge.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “therapeutically effective” applied to dose or amount refers to that quantity of a compound or pharmaceutical composition that is sufficient to result in a desired activity upon administration to a living animal body in need thereof.

The biologically active formulation of the present invention may be administered topically in dosage unit formulations containing conventional non-toxic pharmaceutically acceptable carriers.

With the aid of commonly used solvents, auxiliary agents and carriers, the instant biologically active formulation may be processed into topical preparations, which may be therapeutically applied topically. Representative pharmaceutical compositions comprise an antiacneic composition in conjunction with one or more polyolprepolymers.

Moreover, it is found that the instant biologically active formulations comprising, for example benzoyl peroxide as antiacneic and polyolprepolymer-2 as penetration enhancer, exhibit surprising synergy over traditionally formulated antiacneics. For example, it is shown that the instant biologically active formulation enhances the efficacy of the antiacneic such that its concentration in the ultimate pharmaceutical composition may be halved over traditional formulations, while increasing the penetration of actives and reducing potential irritation side effects.

It is also determined that by micronizing the antiacneic, the concentration in the ultimate pharmaceutical composition may be reduced. This micronization may enhance efficacy through enhanced permeability at a follicular level.

Other suitable pharmaceutical compositions will be immediately apparent to one skilled in the art.

### EXPERIMENTAL PART

The biologically active formulation of the present invention will be better understood in connection with the following examples, which are intended as an illustration of and not a limitation upon the scope of the invention.

### EXAMPLES OF REPRESENTATIVE PHARMACEUTICAL COMPOSITIONS

### Formulation Examples

The following example is given by way of illustration only and is not to be construed as limiting.

Light Acne Treatment (Cosmetic Drug Product)

| | Quantity |
|---|---|
| Active Ingredient: | |
| Benzoyl Peroxide | 5% |
| Other Ingredients: | |
| Water | A |
| C12-15 Alkyl Benzoate | D |
| Cetearyl Alcohol | E |
| Ethoxydiglycol | E |
| Propylene Glycol | E |
| Glyceryl Laurate | E |
| Behenyl Alcohol | E |
| PEG-100 Stearate | E |
| Cyclopentasiloxane | E |
| Polyolprepolymer-2/SMDI Copolymer | F |
| Cetearyl Glucoside | F |
| Phenoxyethanol | F |
| Hydrogenated Lecithin | F |
| Xanthan Gum | F |
| Caprylyl Glycol | F |
| Polysilicone-11 | F |
| Disodium EDTA | G |
| Alpha Bisabolol | G |
| Dimethylacrylamide/Acrylic Acid/Polystyrene Ethyl | G |
| Methacrylate Copolymer | |
| Oryza Sativa (Rice) Bran Extract | G |

Letter Code - To indicate the approximate percentage of each ingredient
A > 50%
B 25.01-50.00%
C 10.01-25.00%
D 5.01-10.00%
E 1.01-5.00%
F 0.11-1.00%
G < 0.10%

### Pharmacology

The biologically active formulations of the instant invention and methods of treating therewith, are characterized by unique and advantageous properties, rendering the “subject matter as a whole”, as claimed herein, unobvious. The pharmaceutical compositions exhibit, in standard accepted reliable test procedures, the following valuable properties and characteristics:

### Methods

### 50 Human Subject Repeat Insult Patch Test Skin Irritation/Sensitization Evaluation (Open Patch)

Objective: Consumer products or raw materials designed for consistent reapplication to areas of the skin may, under proper conditions, prove to be contact sensitizers or irritants in certain individuals. It is the intention of a Repeat Insult Patch Test (RIPT) to provide a basis for evaluation of this irritation/sensitization potential if such exists.

Test Material: Benzoyl Peroxide

Test Material Description: as described in the above-identified pharmaceutical composition of the biologically active formulation.

Handling: Upon arrival at the laboratories, the test material is assigned a unique laboratory code number and entered into a daily log identifying the lot number, sample description, sponsor, date received and tests requested.

Samples are retained for a period of three months beyond submission of final report unless otherwise specified by the sponsor or, if sample is known to be in support of governmental applications, representative retained samples are kept two years beyond final report submission.

Sample disposition is conducted in compliance with appropriate Federal, state and local ordinances.

Test Material Evaluation Prerequisite: Prior to induction of a human test panel, toxicology, microbiology or in-vitro performance spectra may be required to assess the feasibility of commencement as dictated by an Institutional Review Board (IRB) described in Section 3.0.

Sponsor purports that prior to sample submission the following tests were conducted with no adverse results and that the test data are on file on their premises and have not been made available to AMA personnel:
USP or CTFA Preservative Efficacy Test or equivalent
90 Day Accelerated Stability and Container Compatibility Study

Standards for Inclusion in a Study:
Individuals who are not currently under a doctor's care.
Individuals free of any dermatological or systemic disorder which would interfere with the results, at the discretion of the investigator.
Individuals free of any acute or chronic disease that might interfere with or increase the risk of study participation.
Individuals who will complete a preliminary medical history form mandated by the laboratory and are in general good health.
Individuals, who will read, understand and sign an informed consent document relating to the specific type of study they are subscribing. Consent forms are kept on file and are available for examination on the premises of the laboratory only.
Individuals able to cooperate with the Investigator and research staff, willing to have test materials applied according to the protocol, and complete the full course of the study.

Standards for Exclusion from a Study:
Individuals under 18 years of age.
Individuals who are currently under a doctor's care.
Individuals who are currently taking any medication (topical or systemic) that may mask or interfere with the test results.
Subjects with a history of any acute or chronic disease that might interfere with or increase the risk associated with study participation.
Individuals diagnosed with chronic skin allergies.
Female volunteers who indicate that they are pregnant or lactating.

Recruitment: Panel selection is accomplished by advertisements in local periodicals, community bulletin boards, phone solicitation, electronic media or any combination thereof.

Informed Consent and Medical History Forms: An informed consent is obtained from each volunteer prior to initiating the study describing reasons for the study, possible adverse effects, associated risks and potential benefits of the treatment and their limits of liability. Panelists sign and date the informed consent document to indicate their authorization to proceed and acknowledge their understanding of the contents. Each subject is assigned a permanent identification number and completes an extensive medical history form. These forms along with the signed consent forms, are available for inspection on the premises of the laboratory only. Reference 21 CFR Ch. 1 Part 50, Subpart B.

Population Demographics:
Number of subjects enrolled . . . 52
Number of subjects completing study . . . 50
Age Range . . . 18-65
Sex . . . Male . . . 7
   Female . . . 45
Race . . . Caucasian . . . 42
   Hispanic . . . 7
   Asian . . . 3

Equipment:
Acculine Surgical Marking Pen (Accu-line Products, Inc.)
1 ml volumetric syringe without a needle.

Procedure:
Subjects are requested to bathe or wash as usual before arrival at the facility.
0.2 ml or 0.2 g of the test material is dispensed directly onto a designated area of the panelist's back and allowed to air dry.
This procedure is repeated until a series of nine consecutive ‘open patch’ applications have been made for every Monday, Wednesday, and Friday for three consecutive weeks.
In the event of an adverse reaction, the area of erythema and edema is measured. The edema is estimated by the evaluation of the skin with respect to the contour of the unaffected normal skin. Reactions are scored just before applications two through nine and the next test date following application nine. In most instances this is approximately 48 hours after application. Clients are notified immediately in the case of adverse reaction and determination is made as to treatment program if necessary.
Subjects are then given a 10-14 day rest period after which a challenge or retest dose is applied once to a previously unexposed test site. The retest dose is equivalent to any one of the original nine exposures. Reactions are scored 24 and 48 hours after application.
Comparison is made between the nine inductive responses and the retest dose.

Obser vations: No adverse reactions of any kind were noted during the course of this study.

Conclusions: The test material when tested under ‘open patch’ conditions as described herein, may be considered; a NON-PRIMARY IRRITANT and NON-PRIMARY SENSITIZER to the skin according to the reference.

### CONCLUSIONS

In conclusion, from the foregoing, it is apparent that the present invention provides novel, valuable, and unpredictable applications and uses of the biologically active formulations of the present invention, which biologically active formulations comprise an antiacneic according to the present invention, as well as a polyolprepolymer. Methods of treating therewith demonstrate more specifically-enumerated characteristics and advantages.

The present invention is not to be limited in scope by the specific embodiments described herein. Indeed, various modifications of the invention in addition to those described herein will become apparent to those skilled in the art from the foregoing description.

All patents, applications, publications, test methods, literature, and other materials cited herein are hereby incorporated by reference.

We claim:

1. A topical biologically active formulation, comprising a biologically active agent in combination with a penetration enhancer;
wherein the biologically active agent is an antiacneic substance; and
wherein the penetration enhancer is a polyolprepolymer.

2. The topical biologically active formulation of claim 1, wherein the antiacneic substance is benzoyl peroxide.

3. The topical biologically active formulation of claim 1, wherein the antiacneic substance is benzoyl peroxide which has been micronized to 5 μm.

4. The topical biologically active formulation of claim 1, wherein the polyolprepolymer is polyolprepolymer-2.

5. The topical biologically active formulation of claim 1, further comprising another substance effective in preventing and treating acne.

6. A pharmaceutical composition, comprising the topical biologically active formulation of claim 1, alone or in combination with one or more pharmaceutically acceptable additives and/or excipients.

7. The pharmaceutical composition of claim 6, wherein the antiacneic substance is benzoyl peroxide.

8. The pharmaceutical composition of claim 7, wherein the benzoyl peroxide is present in an amount between 0.1 and 20 weight percent.

9. The pharmaceutical composition of claim 8, wherein the benzoyl peroxide is present in an amount between 3 and 10 weight percent.

10. The pharmaceutical composition of claim 6, wherein the penetration enhancing agent is a polyolprepolymer.

11. The pharmaceutical composition of claim 10, wherein the polyolprepolymer is polyolprepolymer-2.

12. The pharmaceutical composition of claim 11, wherein the polyolprepolymer-2 is present in an amount between 0.1 to 20 weight percent.

13. The pharmaceutical composition of claim 12, wherein the polyolprepolymer-2 is present in an amount between 1 to 10 weight percent.

14. The pharmaceutical composition of claim 6, wherein the antiacneic substance is benzoyl peroxide which is present at about 5 weight percent and the polyolprepolymer is polyolprepolymer-2 which is present at about 1 weight percent.

15. The pharmaceutical composition of claim 6, wherein the composition is in the form of a liquid, gel, lotion, cream or ointment.

16. A method of treating or preventing acne, comprising administering to a patient in need of such treatment the topical biologically active formulation of claim 1, which provides the biologically active agent in a therapeutically effective amount.

17. The method of claim 16, wherein the biologically active formulation of claim 1 comprises a therapeutically active amount of benzoyl peroxide.

18. The method of claim 16, wherein the biologically active formulation of claim 1 comprises a therapeutically active amount of benzoyl peroxide in combination with polyolprepolymer-2 as penetration enhancer.
